(19) United States
(12) Patent Application Publication
(10) Pub. No.: US 2006/0154902 A1
(43) Pub. Date: Jul. 13, 2006

(54) Shortening of hospital stay and improving survival in patients with chronic kidney disease

(76) Inventors: Joel Melnick, Wilmette, IL (US); Laura Williams, Gurnee, IL (US); Michael J. Amdahl, Wauconda, IL (US); Steven E. Marx, Hoffman Estates, IL (US); Mark H. O'Brien, Libertyville, IL (US); Angelo Mathes, Montreal (CA)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/524,376

(22) PCT Filed: Aug. 15, 2003
(86) PCT No.: PCT/US03/25780

§ 371 Date: Oct. 14, 2005

(30) Foreign Application Priority Data
Aug. 16, 2002 (US) 60403930
May 13, 2003 (US) 60470117

(51) Int. Cl. A61K 31/59

(52) U.S. Cl. 514/167

(57) ABSTRACT

Formulations containing a Vitamin D compound or analog, such as paricalcitol (Zemplar™) are useful for shortening hospital stays in chronic kidney disease patients with or without hyperparathyroidism. Also disclosed are methods of shortening hospital stays for chronic kidney disease patients with or without hyperparathyroidism, and methods for determining reduction length of hospital stay in chronic kidney disease patients with or without hyperparathyroidism. Titration to serum calcium or serum PTH is avoided by use of the invention.

### CROSS REFERENCE TO RELATED APPLICATIONS

This application claims priority to the provisional Application No. 60/403,930 filed on Aug. 16, 2002 and to the provisional Application entitled Improved Hospitalization Outcomes In Hemodialysis Patients Treated With Paricalcitol, filed May 13, 2003 as attorney docket no. 7066.US.Z1.

### FIELD OF THE INVENTION

This invention is generally directed to formulations containing Vitamin D compounds or Vitamin D analogs, especially paricalcitol, which are useful to shorten hospital stays and improve survival in patients receiving chronic renal replacement therapy. The invention also relates to methods of shortening hospital stays for patients with chronic kidney disease, and methods for determining reduction length of hospital stay in patients with chronic kidney disease.

### BACKGROUND OF THE INVENTION

Approximately half of the thousands of patients receiving chronic renal replacement therapy suffer from secondary hyperparathyroidism, which is often accompanied by skeletal abnormalities, cardiovascular complications, infections and immunoregulatory dysfunction, foot and extremity complications, anemia, or some combination of the foregoing. These patients are at increased risk for fracture calciphylaxis and cardiovascular events, all of which may result in lengthy hospital stays, morbidity, and mortality, which can be magnified by abnormalities in serum calcium and phosphorus. While Vitamin D compounds or analogs can reverse hyperparathyroidism, they can affect calcium and phosphorus homeostasis. Different compounds can modulate serum parathyroid hormone, calcium and phosphorus differently, which may affect morbidity and mortality differently.

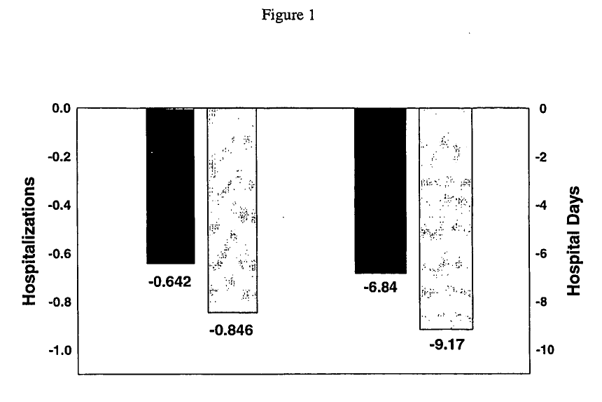

Our recent data (Dobrez, et al, Nephrology, Dialysis and Transplantation, manuscript accepted) have demonstrated that therapy with paricalcitol (commercially available under the Zemplar® mark from Abbott Laboratories, North Chicago, Ill.) associates with lower hospitalizations compared with calcitriol (commercially available under the Calcijex® mark from Abbott Laboratories, North Chicago, Ill.). FIG. 1. presents ordinary least squares models measuring the impact on all-cause hospitalizations in a subset of paricalcitol patients who remained exclusively on the initial vitamin D therapy □ compared to the intent-to-treat population ▪. Negative coefficients reflect fewer hospitalizations and hospital days for paricalcitol compared with calcitriol in both intent-to-treat and monotherapy groups. p<0.0001 for all (n=11,443)

Further, data from others (Teng, et al, New England Journal of Medicine, 2003) have shown that patients receiving paricalcitol experience a significant improvement in survival. These findings are consistent since mortality is linked with morbidity (Lauska and Kryscio, Neurology, 1994; Keller and Potter, Journal of Gerontology, 1994).

However, neither of these studies was able to differentiate whether the benefit of decreased mortality and morbidity reflected improvement of mineral imbalance or an effect of a specific vitamin D therapy. The authors report that a survival benefit did not associate with Vitamin D dose and was independent of baseline serum calcium or phosphorus. We also found no dose response associated with hospitalizations and no significant difference for mean serum calcium and phosphorus between calcitriol and paricalcitol patient groups despite differences in hospitalizations. Further, human studies (Salusky and Goodman, Nephrology, Dialysis and Transplantation, 2002) have suggested that Vitamin D therapy can worsen mortality and morbidity in patients with chronic kidney disease, for example, by causing vascular calcification. Therefore, the medical community has not uniformly endorsed use of Vitamin D compounds in these patients.

Finally, the administration of pharmacological Vitamin D therapy conventionally employs titrating the dose to an effect—correction of PTH and/or serum calcium. Initial doses are based upon patient weight or severity of disease. Subsequent doses, in addition to titration to effect, are monitored to avoid overtreatment, e.g., oversuppression of PTH. All the while, these judicious dose adjustments are achieved while averting side effects. Since overtreatment and side effects due to Vitamin D therapy appear to affect unfavorable outcomes, as mentioned in the previous paragraph, these dose titrations are relied on to optimize therapy.

The majority of the deaths occurring in patients with chronic kidney disease results from cardiovascular, infectious and/or oncologic causes, regardless of serum PTH. It would be advantageous to reduce the morbidity and mortality of these patients. There is therefore an ongoing need for an improved treatment regimen for patients suffering from the effects of chronic kidney disease with or without secondary hyperparathyroidism, which improved treatment regimen results in shorter hospital stays and subsequent improved survival.

### SUMMARY OF THE INVENTION

A first embodiment of this invention, therefore, is directed to formulations containing a Vitamin D compound or analog, especially paricalcitol, which are useful for shortening the hospital stay and improving survival in patients with chronic kidney disease with or without secondary hyperparathyroidism compared to chronic kidney disease patients not treated with a Vitamin D compound or analog.

A second embodiment of this invention is directed to methods of treating patients with a Vitamin D compound or analog, especially paricalcitol, the method providing shortened hospital stays and improving survival in patients with chronic kidney disease with or without hyperparathyroidism. Preferred embodiments of this aspect of the invention do not titrate to serum PTH or serum calcium. Hospital stays and survival are improved compared to chronic kidney patients not treated with a Vitamin D compound or analog.

A third embodiment of this invention is directed to a method for reducing the length of hospital stays for chronic kidney disease patients with or without hyperparathyroidism. According to this aspect of the invention, a therapeutically effective amount of a Vitamin D compound or analog-containing formulation is administered to a chronic kidney disease patient without titrating to serum calcium or serum PTH level. Hospitalizations and hospital days are reduced compared to those for a chronic kidney disease patient not receiving a Vitamin D compound or analog-containing formulation.

Paricalcitol is a preferred Vitamin D compound or analog.

A preferred regimen is equivalent to 4 mcg of paricalcitol or 1 mcg of calcitriol administered three times weekly or 2 mcg of paricalcitol or 0.5 mcg of calcitriol administered daily.

### DETAILED DESCRIPTION OF THE INVENTION

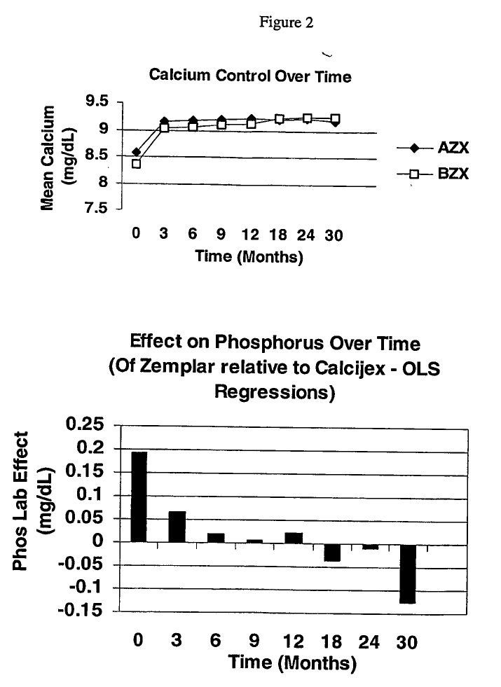

Vitamin D exhibits functions beyond modulation of serum parathyroid hormone, calcium, phosphorus and the resultant bone effects. Vitamin D modulates cell differentiation and proliferation in the cardiovascular and immune system, and in various malignant and pre-malignant tissues. Importantly, we found that these broader effects of Vitamin D are independent of control of serum calcium and phosphorus. As shown in FIG. 2, a historical cohort of 11,340 adult patients, new to hemodialysis, was followed over a 35-month period (January 1999 thru November 2001) using a dialysis provider database. Patients entered the cohort at any time. Vitamin D use was defined by the administration of at least doses of a Vitamin D product. Hospitalizations were identified by documented hospitalized absences from the dialysis clinic and were standardized by patient observation period. ANOVA or Chi-Square tests were used to evaluate differences in baseline characteristics. Univariate tests and negative binomial regression models were used to evaluate hospitalization outcomes: hospital days per year and hospitalizations per year.

Analysis revealed that 2,316 patients with baseline characteristics as identified in Table 1 were treated with paricalcitol (“Par”), 2,299 with calcitriol (“Cal”), and 6,725 did not receive Vitamin D therapy (“No D”). “No D” patients did not receive a placebo. Univariate analyses revealed significant differences at baseline (p<0.0001) among paricalcitol, calcitriol and No D groups, respectively, (as shown in Table 1) in mean age (62 vs. 64 vs. 65), mean iPTH (558 vs. 419 vs. 182), mean calcium (8.4 vs. 8.2 vs. 8.7), mean Ca×P product (44 vs. 41 vs. 44), race (42% vs. 33% vs. 19% African American), co-morbidities (40% vs. 33% vs. 35% blood disorders) and geographic region. There was no difference for baseline phosphorus.

| | Paricalcitol* | Calcitriol* | NoD* | |
|---|---|---|---|---|
| Independent Variables | (n = 2,316) | (n = 2,299) | (n = 6,725) | p-Value |
| Clinical Laboratory Values | | | | |
| Serum PTH (ng/Ml) | 558.4 ± 7.9 | 418.7 ± 6.3 | 181.8 ± 2.5 | <0.0001 |
| Serum Calcium (mg/dL) | 8.45 ± 0.02 | 8.19 ± 0.02 | 8.73 ± 0.01 | <0.0001 |
| Serum Phosphorous (mg/dL) | 5.19 ± 0.04 | 5.03 ± 0.04 | 5.07 ± 0.02 | <0.0001 |
| Calcium × Phosphorus | 43.7 ± 0.3 | 41.0 ± 0.3 | 44.1 ± 0.2 | NS |
| Demographics | | | | |
| Mean Age (years) | 61.8 ± 0.3 | 64.4 ± 0.3 | 65.4 ± 0.2 | <0.0001 |
| Female (%) | 48.9 | 46.8 | 45.6 | 0.021 |
| African American (%) | 42.1 | 33.4 | 18.7 | <0.0001 |
| Co-Morbid Conditions (%)^{†‡} | | | | |
| DM | | | | |
| Adult Onset DM | 48.5 | 51.5 | 52.1 | NS |
| Childhood Onset DM | 3.6 | 3.4 | 3.8 | NS |
| No DM | 37.4 | 35.1 | 34.8 | NS |
| DM Status Unknown | 10.5 | 10.1 | 9.3 | NS |
| Other Endocrine | 43.9 | 41.9 | 42.0 | NS |
| Infectious Disease | 3.9 | 3.0 | 3.0 | NS |
| Neoplasm | 3.9 | 4.3 | 5.3 | NS |
| Hematologic | 40.4 | 33.3 | 35.4 | <0.0001 |
| Mental | 4.5 | 4.1 | 4.8 | NS |
| Nervous System | 10.2 | 8.4 | 9.7 | NS |
| Cardiovascular | 52.3 | 51.2 | 52.0 | NS |
| Respiratory | 6.0 | 5.6 | 6.9 | NS |
| Digestive | 9.5 | 7.7 | 10.1 | NS |
| Genitourinary | 30.7 | 25.3 | 27.7 | NS |
| Pregnancy-Related | 0.3 | 0.0 | 0.1 | NS |
| Skin | 1.5 | 1.7 | 2.1 | NS |
| Muscle and Bone | 5.7 | 3.9 | 6.0 | NS |
| Congenital | 1.7 | 1.4 | 1.4 | NS |
| Trauma/Injury | 9.8 | 7.8 | 9.9 | NS |

DM = diabetes mellitus,
PTH = intact parathyroid hormone
*Column totals for individual categories may exceed 100% due to rounding.
^{†}Per ICD-9 Code
^{‡}Patients may have had more than one condition; totals may exceed 100%.

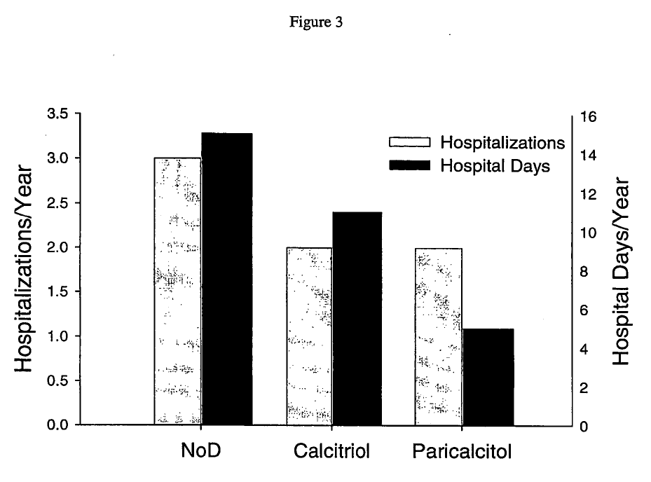

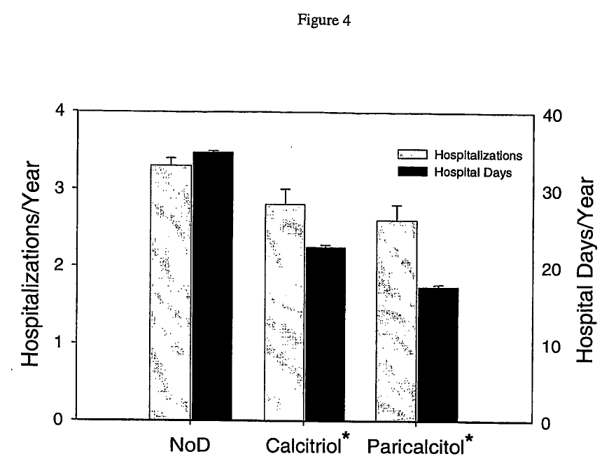

Evaluation of hospitalization endpoints revealed median annual hospitalizations for paricalcitol, calcitriol and No D groups (2 vs. 2 vs. 3) and median days in the hospital per year (5 vs. 11 vs. 15), respectively. As shown in FIG. 3, negative binomial regression analysis revealed that patients who did not receive Vitamin D experienced 59% more hospital days per year compared calcitriol group (p<0.0001) and 17% more annual hospitalizations (p<0.006). However, compared to the paricalcitol group, the No D group experienced 30% more annual hospitalizations and 100% more days per year in the hospital (p<0.0001 for both)(as shown in FIG. 4).

Patients with chronic kidney disease who did not receive Vitamin D experienced more hospitalizations and more days in the hospital compared to those who were treated with either paricalcitol or calcitriol. Furthermore, patients treated with paricalcitol experienced the fewest hospitalizations and days in the hospital, which may reflect additional beneficial effects of Vitamin D compounds and analogs beyond mineral and PTH control.

Suitable patients to be treated according to the invention can have chronic kidney disease with or without hyperparathyroidism. Thus, according to one embodiment, the present invention relates to a method of treating patients by administering formulations containing Vitamin D compounds or analogs. Paricalcitol-containing formulations are preferred. For example, preferred treatment or preventive regimens for patients with chronic kidney disease according to the present invention would administer therapeutically effective Vitamin D compound or analog-containing compositions as a bolus dose orally or intravenously or as a continuous or sustained dose by depot, transdermal or oral routes for a sufficient period to improve survival and/or to decrease morbidity. Suitable delivery forms include but are not limited to tablets or capsules for oral administration, injections, transdermal patches for topical administration (e.g., drug to be delivered is mixed with polymer matrix adhered to or absorbed on a support or backing substrate, e.g. ethylcellulose), depots (e.g., injectable microspheres containing the desired bioactive compounds) and implants.

The formulations can be administered intravenously or orally at least three times weekly. This dose does not require titration to effect—e.g., correction of PTH or serum calcium, in contrast to conventional Vitamin D therapies—since mortality and morbidity are independent to markers of mineral balance. An exemplary preferred minimum administered dose is equivalent to 4 mcg of paricalcitol or 1 mcg of calcitriol administered two to three times weekly or 2 mcg of paricalcitol or 0.5 mcg of calcitriol administered daily. Long term treatment with the formulations of the invention is possible to maintain the benefits without adverse side effects.

What is claimed is:

1. A formulation containing a therapeutically effective amount of at least one Vitamin D compound or analog (for shortening the hospital stay and improving survival in patients with chronic kidney disease with or without hyperparathyroidism compared to chronic kidney disease patients that are not receiving a Vitamin D compound or analog-containing formulation.

2. A formulation according to claim 1, wherein said at least one Vitamin D compound or analog is paricalcitol.

3. A formulation according to claim 1, wherein said at least one Vitamin D compound or analog is calcitriol.

4. A formulation according to claim 1, wherein said formulation is in injectable dosage form.

5. A formulation according to claim 1, wherein said formulation is in oral dosage form.

6. A method of shortening hospital stays and improving survival in chronic kidney disease patients, said method comprising the step of administering a composition containing a therapeutically effective amount of at least one Vitamin D compound or analog without titrating to serum PTH or serum calcium, wherein hospital stays and survival are shortened compared to chronic kidney disease patients not receiving any Vitamin D compound or analog-containing composition.

7. A method according to claim 6, wherein said composition is administered intravenously or orally.

8. A method according to claim 7, wherein said composition is administered at least three times weekly.

9. A method according to claim 8, wherein a minimum administered dose is equivalent to 4 mcg of paricalcitol or 1 mcg of calcitriol administered three times weekly or 2 mcg of paricalcitol or 0.5 mcg of calcitriol administered daily.

10. A method according to claim 6 wherein hospital stay is shortened by at least about seven days annually.

11. A method of treating a chronic kidney disease patient to reduce hospitalizations and hospital days, comprising the step of: administering a therapeutically effective amount of Vitamin D) compound or analog-containing formulation to said patient without titrating to serum calcium or serum PTH level, wherein said hospitalizations and hospital days are reduced compared to those for a chronic kidney disease patient not receiving a Vitamin D compound or analog containing formulation.

12. A method according to claim 11, wherein said patient has hyperparathyroidism.

13. A method according to claim 11, wherein said formulation comprise paricalcitol.

14. A method according to claim 11, wherein said formulation comprises calcitriol.

15. A method according to claim 11, wherein said formulation administered to said patient is equivalent to 4 mcg of paricalcitol or 1 mcg of calcitriol administered three times weekly or 2 mcg of paricalcitol or 0.5 mcg of calcitriol administered daily.

16. A method according to claim 11, wherein said administering step is conducted via injection.

17. A method according to claim 11, wherein said administering step is conducted orally.

18. A method according to claim 11, wherein hospitalization is reduced by at least about 7 days annually.
